(19) United States
(12) Patent Application Publication
(10) Pub. No.: US 2022/0323347 A1
(43) Pub. Date: Oct. 13, 2022

(54) Medicant Delivery Device

(71) Applicant: Mike Sexton, Lake St Louis, MO (US)

(72) Inventor: Mike Sexton, Lake St Louis, MO (US)

(21) Appl. No.: 17/224,206

(22) Filed: Apr. 7, 2021

(51) Int. Cl. A61K 9/00; B65D 65/46; A23K 50/40; A23K 40/30; A61D 7/00

(52) U.S. Cl. CPC A61K 9/0056; B65D 65/463; A23K 50/40; A23K 40/30; A61D 7/00

(57) ABSTRACT

A medicant delivery device for masking taste of a medicant for facilitating oral delivery to an animal includes a shell, which defines an interior space. The shell has a top, which is open for insertion of a medicant therethrough into the interior space. The shell comprises one or more of protein, fat, and vegetable matter, so that the shell is both edible and formable. The shell is at least one of compressible and foldable, proximate to the top, to encase and substantially seal the medicant within the shell. The shell containing the medicant is delivered orally to an animal to medicate the animal.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to delivery devices and more particularly pertains to a new delivery device for masking taste of a medicant for facilitating oral delivery to an animal. The present invention discloses a delivery device comprising an edible, thimble shaped, shell into which a medicant can be positioned and substantially sealed by compression of the shell proximate to a top thereof.

### (2) Description of Related Art Including Information Disclosed Under 37 CFR 1.97 and 1.98.

The prior art relates to delivery devices. Prior art delivery devices may comprise edible envelops, capsules, pockets, jackets, and the like, into which a pill or other medicant can be positioned. What is lacking in the prior art is a delivery device comprising an edible, thimble shaped, shell into which a medicant can be positioned and substantially sealed by compression of the shell proximate to a top thereof.

### BRIEF SUMMARY OF THE INVENTION

An embodiment of the disclosure meets the needs presented above by generally comprising a shell, which defines an interior space. The shell has a top, which is open and configured for insertion of a medicant therethrough into the interior space. The shell comprises one or more of protein, fat, and vegetable matter, so that the shell is both edible and formable. The shell is configured to be at least one of compressed and folded, proximate to the top, to encase and substantially seal the medicant within the shell. The shell containing the medicant is configured for oral delivery to an animal to medicate the animal.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a medicant delivery device according to an embodiment of the disclosure.

FIG. 2 is a top view of an embodiment of the disclosure.

FIG. 3 is a cross-sectional view of an embodiment of the disclosure.

FIG. 4 is an in-use view of an embodiment of the disclosure.

FIG. 5 is an in-use view of an embodiment of the disclosure.

### DETAILED DESCRIPTION OF THE INVENTION

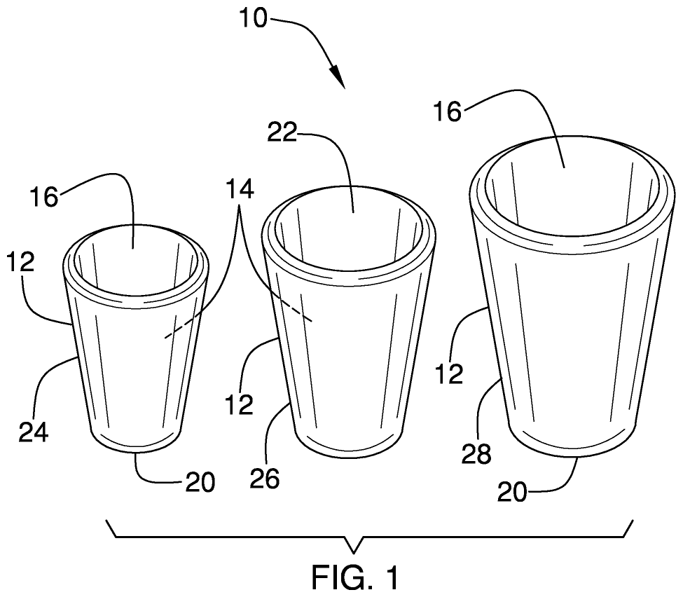

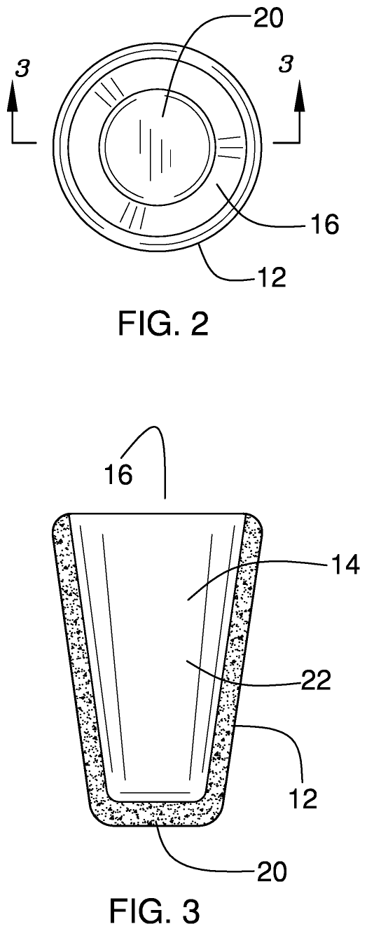

With reference now to the drawings, and in particular to FIGS. 1 through 5 thereof, a new delivery device embodying the principles and concepts of an embodiment of the disclosure and generally designated by the reference numeral 10 will be described.

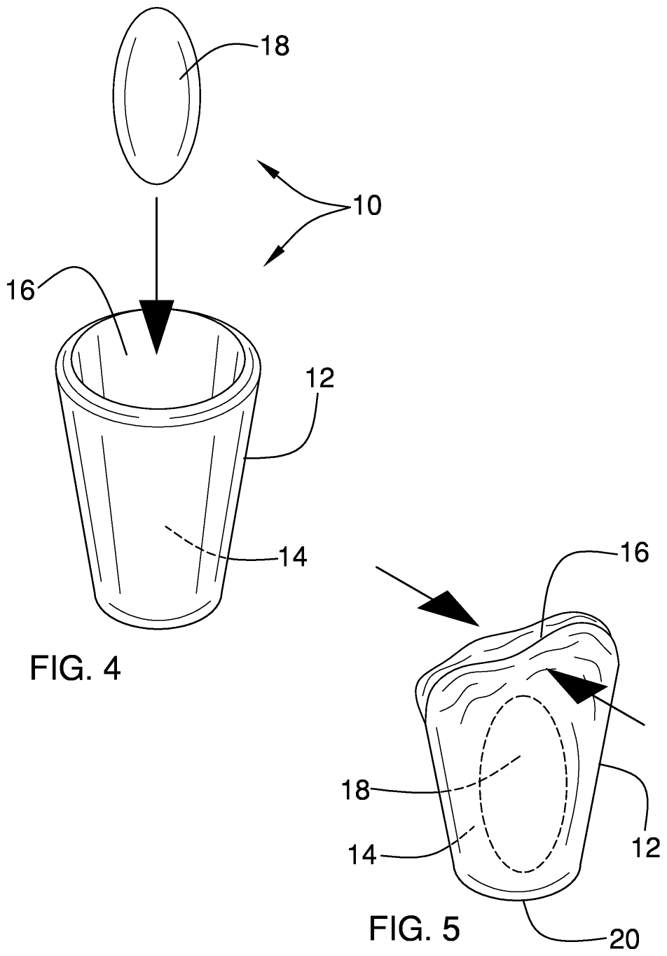

As best illustrated in FIGS. 1 through 5, the medicant delivery device 10 generally comprises a shell 12, which defines an interior space 14. The shell 12 has a top 16, which is open and configured for insertion of a medicant 18 therethrough into the interior space 14, as shown in FIG. 4. The shell 12 comprises one or more of protein, fat, and vegetable matter so that the shell 12 is both edible and formable. The shell 12 is configured to be at least one of compressed and folded, proximate to the top 16, to encase and substantially seal the medicant 18 within the shell 12, as shown in FIG. 5. The shell 12 containing the medicant 18 is configured for oral delivery to an animal to medicate the animal.

The shell 12 may comprise meat, such as, but not limited to, one or more of pepperoni and chicken. The shell 12 also may comprise vegetable matter. Animals, and canines in particular, can be sensitive to tastes and smells of medicants 18, whether formulated as pills, capsules, powders, pastes, syrups, or the like. The animal's sensitive taste and smell can cause difficulties in medicating the animals. The present invention facilitates oral delivery of medicants 18 to animals by providing a means of masking the taste and smell of medicants 18 via the shell 12. The shell 12 comprises meat or vegetable matter, which can be selected specifically to appeal to a particular animal's tastes.

The shell 12 is one of a plurality of shells 12, with each shell 12 being conically frustrum shaped so that the top 16 is circumferentially larger than a bottom 20 of the shell 12. The shells 12 being conically frustrum shaped allows them to be nestable, which is convenient for storage prior to use.

The shell 12 is one of a set of shells 12, with each of the shells 12 of the set of shells 12 having an interior space 14 of a respective volume 22. The set of shells 12 thus comprises shells 12 having interior spaces 14 of a variety of volumes 22, as shown in FIG. 1. A respective one of the shells 12 is configured to have positioned therein a medicant 18 sized complementarily to its interior space 14.

The set of shells 12 may comprise a small shell 24, a medium shell 26, and a large shell 28, as shown in FIG. 1. The small shell 24, the medium shell 26, and the large shell 28 are sized to have positioned therein medicants 18 for medicating animals of less than approximately 9.0 kg, animals of more than 9.0 kg and less than approximately 22.0 kg, and animals of more than approximately 22.0 kg, respectively. The present invention also anticipates the set of shells 12 comprising shells 12 of other sizes, such as shells 12 sized for narrower ranges of animal weights.

In use, a shell 12 sized appropriately for the animal to be medicated is selected and the medicant 18 is inserted into the interior space 14. The shell 12 is pinched closed, and potentially folded, to encase and substantially seal the medicant 18 within the shell 12. The shell 12 containing the medicant 18 then is fed to the animal to effect medicant 18 delivery.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medicant delivery device comprising a shell defining an interior space, the shell having a top, the top being open, wherein the top is configured for insertion of a medicant therethrough into the interior space, the shell comprising one or more of protein, fat, and vegetable matter, such that the shell is edible and formable, wherein the shell is configured for at least one of compressing and folding, proximate to the top, for encasing and substantially sealing the medicant within the shell, wherein the shell containing the medicant is configured for oral delivery to an animal for medicating the animal.

2. The medicant delivery device of claim 1, wherein the shell is conically frustrum shaped.

3. The medicant delivery device of claim 1, wherein the shell comprises meat.

4. The medicant delivery device of claim 3, wherein the shell comprises one or more of pepperoni and chicken.

5. The medicant delivery device of claim 1, wherein the shell comprises vegetable matter.

6. The medicant delivery device of claim 1, wherein the shell is one of a plurality of shells, each shell being conically frustrum shaped, such that the top is circumferentially larger than a bottom of the shell, and such that the shells are nestable.

7. The medicant delivery device of claim 1, wherein the shell is one of a set of shells, each of the shells of the set of shells having an interior space of a respective volume, such that the set of shells comprises shells having interior spaces of a variety of volumes, wherein a respective one of the shells is configured for positioning of a medicant sized complementarily to the interior space of the respective one of the shells.

8. The medicant delivery device of claim 7, wherein the set of shells comprises a small shell, a medium shell, and a large shell, with the small shell, the medium shell, and the large shell being sized for positioning of medicants for medicating animals of less than approximately 9.0 kg, animals of more than 9.0 kg and less than approximately 22.0 kg, and animals of more than approximately 22.0 kg, respectively.

9. A medicant delivery system comprising:
a medicant intended for oral delivery to an animal for medicating the animal; and
a shell defining an interior space, the shell having a top, the top being open, such that the top is positioned for insertion of the medicant therethrough into the interior space, the shell comprising one or more of protein, fat, and vegetable matter, such that the shell is edible and formable, wherein the shell is configured for at least one of compressing and folding, proximate to the top, for encasing and substantially sealing the medicant within the shell, wherein the shell containing the medicant is configured for oral delivery to the animal.

10. A medicant delivery device comprising a shell defining an interior space, the shell having a top, the top being open, wherein the top is configured for insertion of a medicant therethrough into the interior space, the shell comprising one or more of protein, fat, and vegetable matter, such that the shell is edible and formable, wherein the shell is configured for at least one of compressing and folding, proximate to the top, for encasing and substantially sealing the medicant within the shell, wherein the shell containing the medicant is configured for oral delivery to an animal for medicating the animal, the shell being conically frustrum shaped, the shell comprising meat, the shell comprising one or more of pepperoni and chicken, the shell comprising vegetable matter.
